(19) United States
(12) Patent Application Publication
(10) Pub. No.: US 2003/0031590 A1
(43) Pub. Date: Feb. 13, 2003

(54) Apparatus for measuring amount of hemodialysis

(76) Inventor: Han Chul Park, Kwangju (KR)

Correspondence Address: JACOBSON, PRICE, HOLMAN & STERN, PROFESSIONAL LIMITED LIABILITY COMPANY, 400 Seventh Street. N.W., Washington, DC, 20004, US

(21) Appl. No.: 09/925,690

(22) Filed: Aug. 10, 2001

(51) Int. Cl. A61M 1/36; A61M 37/00; C02F 1/44

(52) U.S. Cl. 422/44; 604/5.01; 210/646

(57) ABSTRACT

An apparatus for measuring an amount of hemodialysis is disclosed. The apparatus comprises a rotation detecting unit for detecting the number of rotations of a dialysis blood pump, the pump being rotated to pump blood; a counting unit for counting the number of rotations of the dialysis blood pump by receiving a rotation detecting signal from the rotation detecting unit; and a display unit for displaying a value counted by the counting unit to determine the entire amount of hemodialysis based on the amount of hemodialysis per unit rotation of the dialysis blood pump.

### BACKGROUND OF THE INVENTION

1. Field of the Invention

The present invention relates to an appratus for measuring an amount of hemodialysis dose, and more particularly, to an appratus capable of easily determining an amount of hemodialysis dose.

2. Background of the Related Art

The use of hemodialysis is to treat kidney failure patients by removing biological wastes accumulted in those patients through an artificial kidney dialyser. In case that human kidneys cannot carry out the function of removing wastes normally produced in the body, such as urea, creatinine, potassium, sodium, water, and the like, the life of the patients may be threatended to death. One of the ways of removing those substances and supplying depleted substances, such as bicarbonate, is hemodialysis treatment.

The key part of hemodialysis system is the artificial kidney dialyser which contains semipermeable membrane. On one side of the membrane, blood from the patient flows and on the other side, dialysate, which is equvalent to an ideal body fluid, flows. Through the membrane pores substances are exchanged by diffusion and convection. The blood-side of the membrane is connected from and to the patient by tubings, and the dialysate-side, from and to the dialysate supplyer equipment. In order to assure measured amount of blood flow, a rotating blood pump is placed on the tube from the patient.

In order to assure enough blood flow rate, one or other blood access devise is constructed on the arm or the neck of the patient.

FIG. 1 is a perspective view illusrating the construction of a prior hemodialysis system 100. The operation will now be explained with reference to FIG. 1. A needle of a blood-letting catheter 111 is inserted into a vascular access 210 of the patient 200. The blood to be cleaned flows through catheter 111 into an inlet port 131 of a dialyzer 130 in a speed properly regulated by a blood pump 140, and is run along the blood-side dialysis membrane.

The dialyser membrane is semipermeable and usually constructed in tube-shape, thin enough to be called as hollow-fiber. Blood runs inside of the tube and dialysate runs outside of the tubal wall, usually counter current. Since the tubal wall is semipermeable, molecular exchanges occur through the wall of the tube. Actually many hollow-fiber tubes are fixed in paralel as a bundle, and the fiber bundle is incased in a plastic case, which allows dialysate path.

The fresh dialysate is supplied from a controller tank 120 of a dialysate supplyer equipment and the waste fluid returns through a discharging line 122, and is discarded.

Meanwhile, the purified blood by the dialyzer returns through the returning catheter 112 to the patient 200.

One important element of improving dialysis treatment is to monitor dialysis dose (in real time), so that proper and enough amount of treatment can be given each time.

However, in case of undergoing dialysis treatment using prior apparatus, because there is no appratus for numerically displaying the exact amount of hemodialysis dose, the amount of hemodialysis is set by blood flow rate and dialysis time by past esperience; these two parameters are confounding and may be inaccurate especially when the blood pump is manipulated to decrease or increase the flow rate during the treatment. Manipulations of blood flow occur not infrequenly and unexpectedly because of various reasons, such as patient's intolerance. If dialysis is terminated at a preset time, the amount of dialysis will be suboptimal.

While the dialysate is upwardly transported along an outer side of the hollow fiber of the semi-permeable membrane in the dialyzer 130, the impurities are transported through the membrane and are dissolved in the dialysate. The dialysate containing the impurities removed from the blood is discarded through a discharging line 122.

Meanwhile, the impure blood supplied to the dialyzer through the out-going catheter 111 is cleaned through the hollow fiber, and is returned to the patient 200 through the venous catheter 112.

One essential element for improving dialysis patent care is the effective and speedy detection and measurement of the recirculation. For example, the dialysis treatment time on an interested patient is accurately calculated. The improper dialysis treatment may cause an adverse effect to produce to the patient. Accordingly, it is important to undergo the dialysis in an amount of the recirculation suitable to the patient.

However, in case of undergoing the dialysis using the prior dialyzer, because there is no an apparatus for numerically displaying the exact amount of the recirculation, the amount of the recirculation is measured roughly with ones experience in view of the relationship of the time and the speed of the dialysis blood pump. Accordingly, in case that a nurse carries out the treatment of the dialysis, after a determined time is lapsed, the nurse completes the dialysis.

In addition, during the dialysis, the patient changes the speed of the dialysis blood pump sometimes, because of an inconvenience. At that time, it is very difficult to presume the amount of the recirculation by reference to the dialysis time only.

Therefore, there is a problem in that it is difficult to confirm the amount of the recirculation accurately.

### SUMMARY OF THE INVENTION

Accordingly, the present invention is directed to an apparatus for measuring an amount of hemodialysis that substantially obviates one or more problems due to limitations and disadvantages of the related art.

An object of the present invention is to provide an apparatus for measuring an amount of hemodialysis capable of easily determining the amount of recirculation in a patient undergoing a dialysis treatment.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve the object and other advantages and in accordance with the purpose of the invention, as embodied and broadly described herein, an apparatus for measuring an amount of hemodialysis comprises: a rotation detecting unit for detecting the number of rotations of a dialysis blood pump, the pump being rotated to pump blood; a counting unit for counting the number of rotations of the dialysis blood pump by receiving a rotation detecting signal from the rotation detecting unit; and a display unit for displaying a value counted by the counting unit to determine the entire amount of hemodialysis based on the amount of hemodialysis per unit rotation of the dialysis blood pump.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 illustrates the construction of a prior dialyzer;

FIG. 2 illustrates a dialyzer provided with an apparatus for measuring an amount of hemodialysis according to the present invention;

FIG. 3 illustrates a cross section of an apparatus for measuring an amount of hemodialysis according to the present invention; and

FIG. 4 illustrates a block diagram of an apparatus for measuring an amount of hemodialysis according to the present invention.

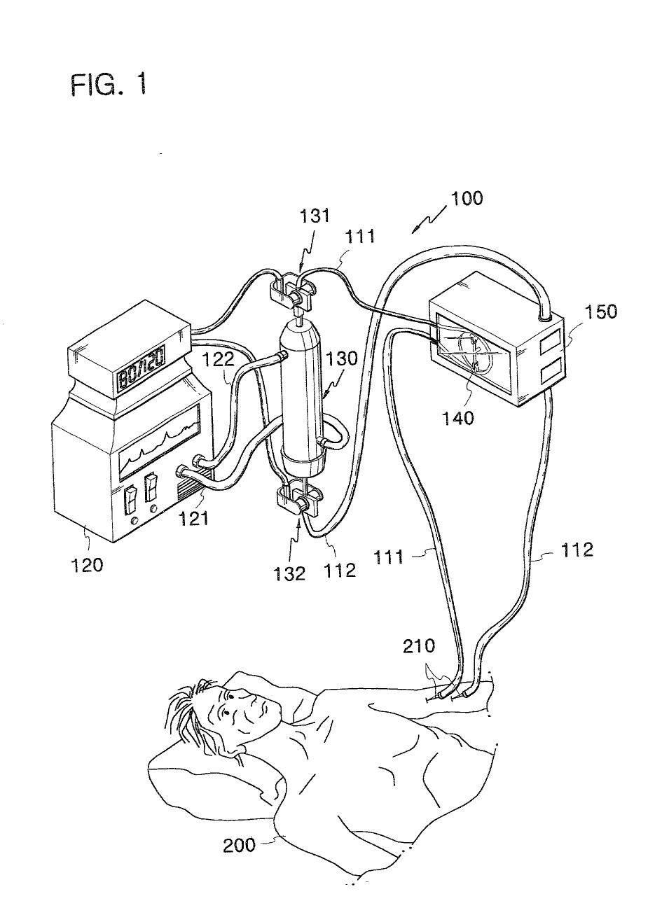

### DETAILED DESCRIPTION OF THE PREFERRED EMBODIMENTS

Reference will now be made in detail to the preferred embodiment of the present invention, examples of which are illustrated in the accompanying drawings.

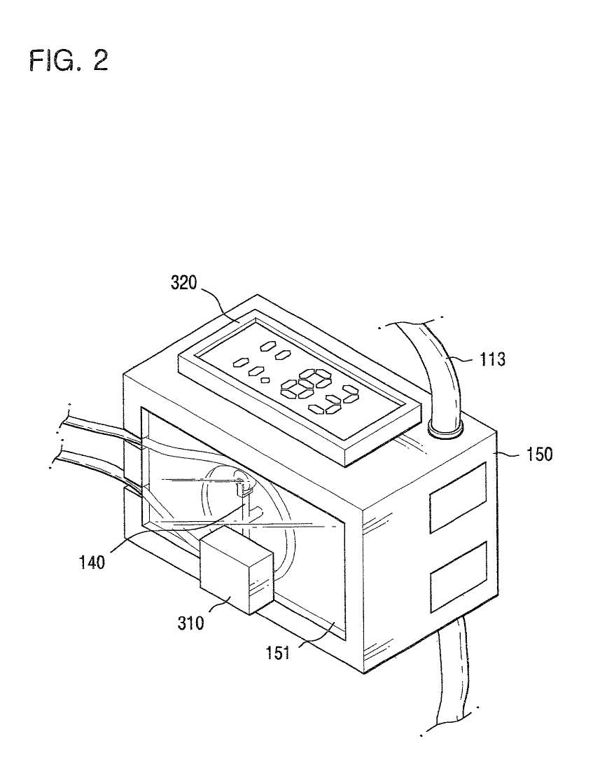

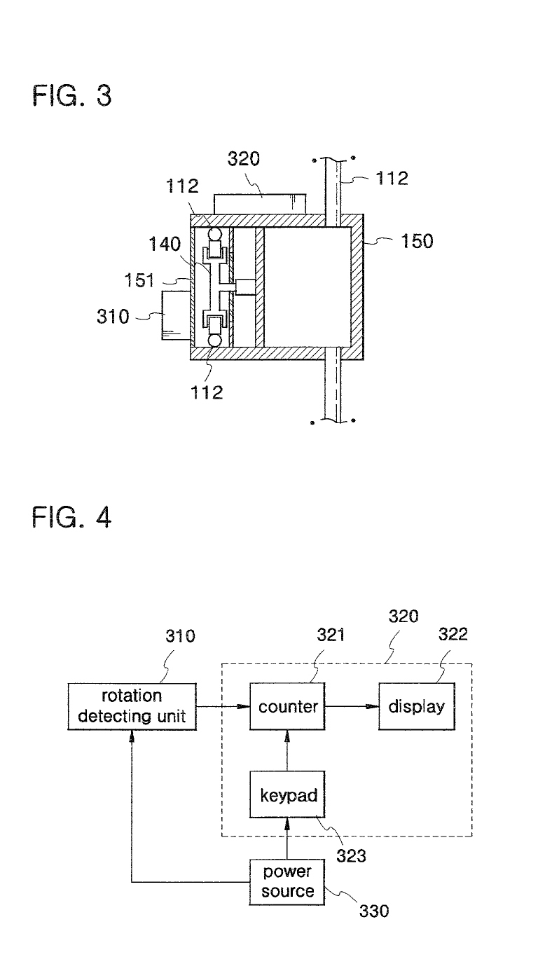

FIG. 2 is a perspective view illustrating a dialyzer provided with an apparatus for measuring an amount of hemodialysis according to the present invention, and FIG. 3 is a cross sectional view of the dialyzer provided with the apparatus for measuring an amount of hemodialysis. The dialyzer comprises a case 150 having a dialysis blood pump 140 for pumping a blood, and a rotation detecting unit 310 is attached to the case to detect the number of rotations. A counting unit 320 is attached to an upper surface of a case 150, and displays the number of rotations on a display portion by receiving a rotation detecting signal from the rotation detecting unit 310.

The rotation detecting unit 310 is positioned on a side of the case 150 as shown in FIG. 3, but the rotation detecting unit 310 may be positioned on any point of the case, on which the rotation detecting unit can detect the rotations. In addition, the counting unit 320 is positioned on the upper surface of the case, but the position thereof may be changed if necessary.

Referring to FIG. 4 showing a block diagram of the apparatus for measuring an amount of hemodialysis according to the present invention, the apparatus includes the rotation detecting unit 310, the counting unit 320, and a power source 330 for applying the power to the rotation detecting unit and the counting unit.

The rotation detecting unit 310 may comprise various kinds of rotation detecting sensors. Because the rotation detecting unit is attached to a front window 151 of the case 150 to detect the number of rotations of the dialysis blood pump, the rotation detecting unit comprises a light sensor provided in a single circuit block, the light sensor consisting of light emitting element for emitting a light to detect the number of rotations of any object, and a light receiving element for receiving a light reflected from the object to output an electrical signal.

Further, the light sensor may be provided in such a manner that the light emitting element is provided on the front window of the case, while the light receiving element is positioned on an inside of a pump chamber opposed to the light emitting element. The light receiving element outputs a counting signal by periodically receiving the light emitted from the light emitting element, which is periodically interrupted by the rotating pump.

Of course, the light emitting element is provided on the front window 310 of the case, while the light receiving element is positioned on an inside of the pump chamber, with the pump and the front window being interposed between the light receiving element and the light receiving element. With the construction, the light emitted from the light emitting element is periodically interrupted by the rotating pump, and the light receiving element receives periodically the light interrupted by the pump to produce an electrical signal.

The counting unit 320 includes a counter 321 for the electrical signal outputted when the rotation detecting unit 310 detects the rotations of the dialysis blood pump, a display 322 for displaying the number of rotations of the dialysis, and a keypad 323 for setting the operation of the counter and the display. The display 323 comprises a light emitting diode, a liquid crystal display or the like

The operation of the apparatus constructed as described above will now be explained in detail. In order to begin to undergo the dialysis, when a start switch of the power source 330 is turned on, a power is applied to the rotation detecting unit 310. And then, the light emitting element of the rotation detecting unit 310 emits the light to the light receiving element to detect the rotation of the dialysis blood pump. In addition, the power is applied to the counter 321 and the display, and the counter is reset to zero and zero is displayed on the display by operating the keypad 323. If the dialysis blood pump 140 rotates to begin to undergo the dialysis. Upon rotating the dialysis blood pump 140, the rotation detecting unit 310 produces the electrical signal, and the counter 321 counts the signal, so that the counted signal is displayed in a type of an Arabic numeral on the display 322. A dialysis operator can see the number of rotations through the Arabic numeral displayed on the display 322.

Since the amount of hemodialysis per one rotation of the dialysis blood pump can be calculated, it will know the entire amount of hemodialysis by multiplying the number of rotations of the dialysis blood pump by the amount of hemodialysis per one rotation of the dialysis blood pump.

Although there are not shown in figures, the apparatus according to the present invention may further comprises a unit for calculating an amount of hemodialyis for calculating the entire amount of hemodialysis by multiplying a counted value inputted from the counter 321 by a pre-stored amount of hemodialysis per one rotation of the dialysis blood pump. With the construction, if the rotation signal of the dialysis blood pump 140 is inputted from the counter 321, the blood recirculation calculating unit calculates the present amount of hemodialysis based on the predetermined amount of hemodialysis per one rotation, so that the result is displayed on the display, thereby allowing the operator to know the amount of hemodialysis easily.

In addition, although there are not shown in figures, the apparatus according to the present invention may further comprises a controller, by which if the amount of hemodialysis is reached to the predetermined amount of hemodialysis, a signalling bell can be ringed or the operation of the dialyzer is stopped. The number of rotations of the dialysis blood pump provided in the case is set in the controller through the keypad 323. If the number of rotations inputted to the controller from the counter is consistent with the preset number of rotations, the controller sets a bell on or the dialysis blood pump to stop, so that the operator may easily operate the dialysis.

The forgoing embodiment is merely exemplary and are not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

What is claimed is:

1. An apparatus for measuring an amount of hemodialysis, the apparatus comprising:
a rotation detecting unit for detecting the number of rotations of a dialysis blood pump, the pump being rotated to pump blood;
a counting unit for counting the number of rotations of the dialysis blood pump by receiving a rotation detecting signal from the rotation detecting unit; and
a display unit for displaying a value counted by the counting unit to determine the entire amount of hemodialysis based on the amount of hemodialysis per unit rotation of the dialysis blood pump.

2. The apparatus as claimed in claim 1, wherein the rotation detecting unit comprises a light emitting element for emitting a light to the rotating dialysis blood pump, and a light receiving element for receiving a light reflected from the dialysis blood pump to output an electrical signal.

3. The apparatus as claimed in claim 1, wherein the rotation detecting unit comprises a light emitting element for emitting a light to the rotating dialysis blood pump, and a light receiving element for outputting a counting signal by periodically receiving the light emitted from the light emitting element, or the light periodically interrupted by the dialysis blood pump.

4. The apparatus as claimed in claim 1, further comprising a keypad for presetting the number of rotations of the dialysis blood pump; and a controller for giving a signal or stopping the dialysis blood pump by comparing the number of rotations inputted from the counting unit with a predetermined amount of hemodialysis inputted by the keypad.

5. The apparatus as claimed in claim 1, further comprising a unit for calculating the amount of hemodialysis for calculating a present amount of hemodialysis based on predetermined amount of hemodialysis per unit rotation of the dialysis blood pump by receiving the counting signal of the dialysis blood pump, and outputting the result to the display unit.
